SEQUENCE LISTING (1 sequence)

SEQ ID NO: 1 - protein, 747 aa, Human
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

(19) United States
(12) Patent Application Publication
(10) Pub. No.: US 2019/0079075 A1
(43) Pub. Date: Mar. 14, 2019

(54) PHARMECEUTICAL COMPOSITION FOR LUNG CANCER TREATMENT AND METHODS FOR PROVIDING INFORMATION AND SCREENING

(71) Applicant: DONG-A UNIVERSITY RESEARCH FOUNDATION FOR INDUSTRY-ACADEMY COOPERATION, Busan (KR)

(72) Inventor: Tae-Hong KANG, Busan (KR)

(21) Appl. No.: 15/743,092

(22) PCT Filed: Jan. 6, 2016
(86) PCT No.: PCT/KR2016/000077

§ 371 Date: Jan. 9, 2018

(30) Foreign Application Priority Data
Jul. 30, 2015 (KR) 10-2015-0108209

(51) Int. Cl. G01N 33/50; G01N 33/574; A61K 31/366; A61K 31/05; A61K 31/404; A61K 31/7068

(52) U.S. Cl. CPC G01N 33/5011; G01N 33/57423; A61K 31/7068; A61K 31/05; A61K 31/404; A61K 31/366

(57) ABSTRACT

A pharmaceutical composition containing a lung cancer therapeutic agent and an SIRT inhibitor may minimize the expression of the resistance of lung cancer cells against the lung cancer therapeutic agent, thereby exerting an excellent lung cancer therapeutic effect. The treatment of lung cancer cells with a lung cancer therapeutic agent and the measurement of a level of SIRT1 expression may provide information for determining whether lung cancer cells have developed chemoresistance to a lung cancer therapeutic agent, and may screen lung cancer therapeutic agents.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2016/000077, filed Jan. 6, 2016, which claims priority to the benefit of Korean Patent Application No. 10-2015-0108209 filed in the Korean Intellectual Property Office on Jul. 30, 2015, the entire contents of which are incorporated herein by reference.

### BACKGROUND

### Technical Field

The present invention relates to a pharmaceutical composition for lung cancer treatment, a method for providing information for the same, and a method for screening the same.

### Background Art

An ultimate goal in cancer therapy is to devise individually tailored treatment plans that target growth-promoting pathways and circumvent drug resistance. In general, tumor cells acquire resistance by manipulating biochemical mechanisms that reduce pharmacokinetics or by acquiring additional alterations in DNA damage response pathways. Hence, an understanding of these processes is important for predicting treatment response and for the development of novel treatment strategies for chemoresistance. Most chemotherapeutics rely for their anticancer activity on induction of a DNA damage response to promote the apoptotic pathway. However, DNA repair pathways counteract this effect by repairing damaged DNA and restoring it to normal status. Cisplatin, carboplatin, and oxaliplatin are platinum-based drugs for treatment of many types of cancers, including head and neck, testicular, ovarian, cervical, lung, colorectal, and relapsed lymphoma. The cytotoxicity of platinating agents is thought to be due to the platinum intrastrand crosslink that forms on DNA, such as Pt-GpG adduct. Resistance can be caused by a number of cellular adaptations, including reduced uptake, inactivation by intracellular antioxidants, and increased DNA repair capacity, especially increased DNA repair capacity (NER capacity) is an important indicator of success of chemotherapy.

However, changes in nucleotide excision repair (NER) kinetics cannot be precisely investigated due to limitations of analytical tools or the like, and it is necessary to study how to suppress changes in NER kinetics to decrease the chemoresistance of cancer cells to anticancer drugs.

### SUMMARY

It is an objective of the present invention to provide a pharmaceutical composition for lung cancer treatment, which is capable of reducing the chemoresistance of lung cancer cells to a lung cancer therapeutic agent.

It is another objective of the present invention to provide a method for providing information for determining whether lung cancer cells have developed chemoresistance to a lung cancer therapeutic agent.

It is still another objective of the present invention to provide a method for screening lung cancer therapeutic agents, which is capable of identifying a lung cancer therapeutic agent with low chemoresistance.

1. A pharmaceutical composition for lung cancer treatment containing a lung cancer therapeutic agent and an SIRT1 inhibitor.

2. The pharmaceutical composition for lung cancer treatment described in 1, wherein the lung cancer therapeutic agent is one or more selected from the group consisting of cisplatin, carboplatin, oxaliplatin, and gemcitabine. 3. The pharmaceutical composition for lung cancer treatment described in 1, wherein the SIRT1 inhibitor is one or more selected from the group consisting of EX527, sirtinol, tenovin-1, tenovin-6, cambinol, salermide, resveratrol, and CAY10602.

4. A method for providing information for determining whether lung cancer cells have developed chemoresistance to a lung cancer therapeutic agent, which includes the processes of treating the lung cancer cells with the lung cancer therapeutic agent and measuring the level of SIRT1 expression.

5. The method for providing information for determining whether lung cancer cells have developed chemoresistance to a lung cancer therapeutic agent described in 4, wherein the lung cancer therapeutic agent is one or more selected from the group consisting of cisplatin, carboplatin, oxaliplatin, and gemcitabine.

6. The method for providing information for determining whether lung cancer cells have developed chemoresistance to a lung cancer therapeutic agent described in 4, which further includes the processes of treating the same lung cancer cells with the same lung cancer therapeutic agent for a predetermined time period and then measuring the level of SIRT1 expression once more; and comparing the measured levels of SIRT1 expression.

7. The method for providing information for determining whether lung cancer cells have developed chemoresistance to a lung cancer therapeutic agent described in 4, which further includes the processes of treating the same lung cancer cells with a plurality of lung cancer therapeutic agents and then measuring the level of SIRT1 expression for each lung cancer therapeutic agent; and comparing the measured levels of SIRT1 expression.

8. A method for screening lung cancer therapeutic agents, which includes the processes of treating lung cancer cells with a plurality of lung cancer therapeutic agents and then measuring the level of SIRT1 expression for each lung cancer therapeutic agent;

and comparing the measured levels of SIRT1 expression.

9. The method for screening lung cancer therapeutic agents described in 8, which identifies a lung cancer therapeutic agent associated with the lowest level of SIRT1 expression as a lung cancer therapeutic agent with the lowest chemoresistance. The pharmaceutical composition for lung cancer treatment according to the present invention can minimize the chemoresistance of lung cancer cells to a lung cancer therapeutic agent. Therefore, the lung cancer therapeutic agent can be 100% effective without inducing chemoresistance and exhibit excellent lung cancer treatment efficacy.

The method for providing information for determining whether lung cancer cells have developed chemoresistance to a lung cancer therapeutic agent according to the present invention can provide information on whether lung cancer cells have developed chemoresistance to a specific lung cancer therapeutic agent. In this way, the method provides information that can be a useful reference in determining time to replace an anticancer agent or identifying an anticancer agent with low chemoresistance.

The method for screening lung cancer therapeutic agents according to the present invention makes it possible to identify lung cancer therapeutic agents to which lung cancer cells develop less chemoresistance. In this way, the method enables the selection of a lung cancer therapeutic agent with low chemoresistance to maximize the effectiveness of lung cancer treatment.

### BRIEF DESCRIPTION OF DRAWINGSFIGS

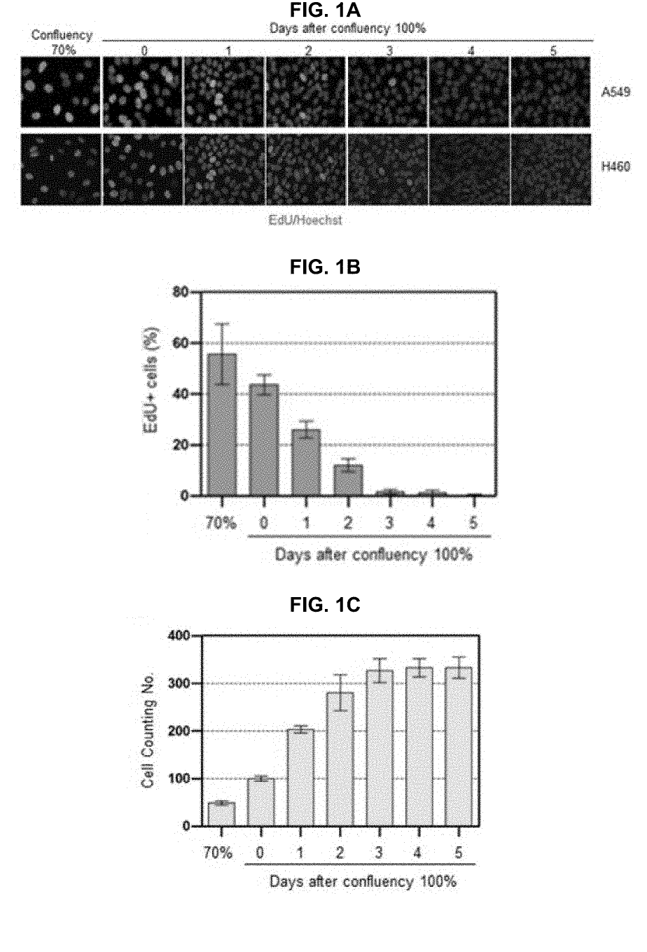

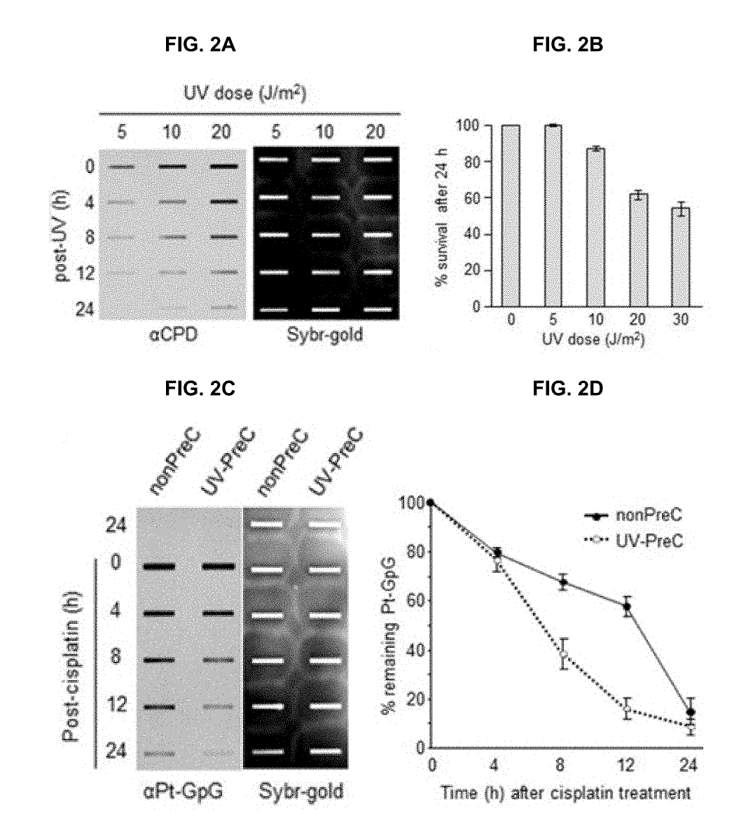

1A to 1C and 2A to 2D show that preconditioning cells with UV irradiation facilitates subsequent repair of cisplatin-induced damage. FIG. 1A shows that A549 and H460 lung carcinoma cells were grown to the indicated density 70% or 100%. The 100% confluency is designated at the time when there is no space among the cells. EdU was added 2 h before fixation at the indicated culture density and days after 100% confluent. FIG. 1B shows that EdU-positive A549 cell numbers were counted among 1000 cells. FIG. 1C shows that the number of A549 cells in at day 0 of 100% confluent was designated as 100 control. The cell numbers from the other samples were plotted as relative values compared to control. The bars and error bars represent the mean±s.d (n=3). FIG. 2A shows that A549 cells irradiated with the indicated UV doses were allowed to carry out repair for the indicated times, followed by isolation of genomic DNA and immunoslot blotting analysis to detect residual cyclobutane pyrimidine dimers (CPDs). After immunoslot blotting, the membrane was counterstained with SYBR-Gold for a loading control of genomic DNA. FIG. 2B shows that cell viability after UV irradiation was assessed by fluorescence-based cell viability assay. Constitutive protease activity within live cells was measured using a fluorogenic and cell permeable peptide substrate using CellTiter-Fluor Cell Viability Assay kit. The fluorescent signal obtained from mock-treated cells was designated as 100 and the relative values obtained from UV-exposed cells were plotted. The bars and error bars represent the mean±s.d (n=3). FIG. 2C shows the removal rates of platinum-GpG (Pt-GpG) from nonpreconditioned (nonPreC) or UV-preconditioned (UV-PreC) cells. Cells were either mock-treated (nonPreC) or 5 J/m² of UV-treated (UV-PreC) and kept for 24 h and followed by 10 μM of cisplatin treatment for 2 h and then culture medium was changed to wash out residual cisplatin in the medium. Recovery times were allowed for the indicated times and genomic DNAs obtained from each time point were assessed by immunoslot blotting using Pt-GpG-specific monoclonal antibody. FIG. 2D shows the quantitative analysis for FIG. 2C. The bars and error bars represent the mean±s.d from three independent experiments.

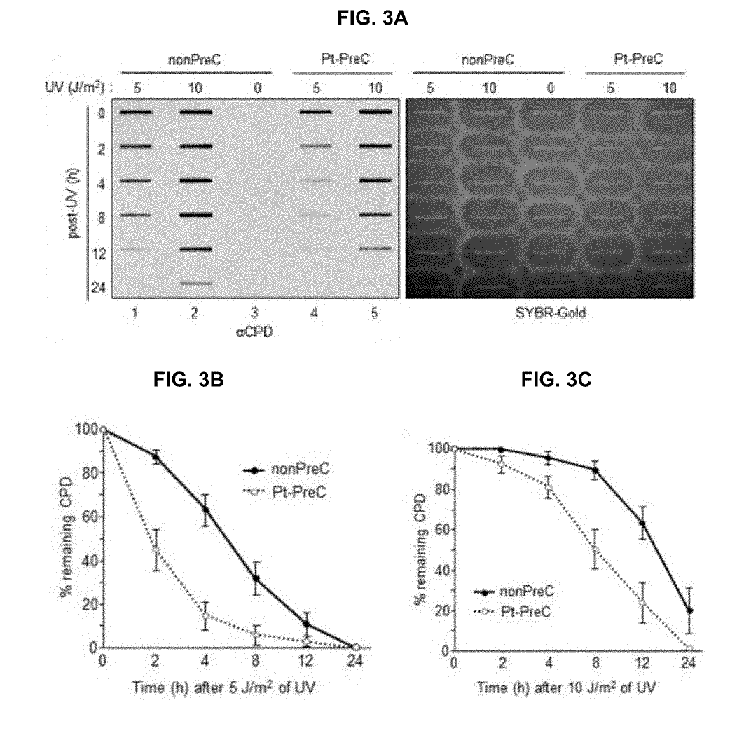

FIGS. 3A to 3C show the enhanced UV-induced CPD repair activity following Pt-PreC. FIG. 3A shows that the residual CPDs in genomic DNA of nonpreconditioned cells (nonPreC) or cells preconditioned with 5 μM of cisplatin (Pt-PreC) were assessed by immunoslot blotting. After immunoslot blotting the membrane was counterstained with SYBR-Gold for a loading control of genomic DNA. 5 J/m² (FIG. 3B) or 10 J/m² (FIG. 3C) of UV-induced CPD repair kinetics were measured from cells conditioned with nonPreC or Pt-PreC.

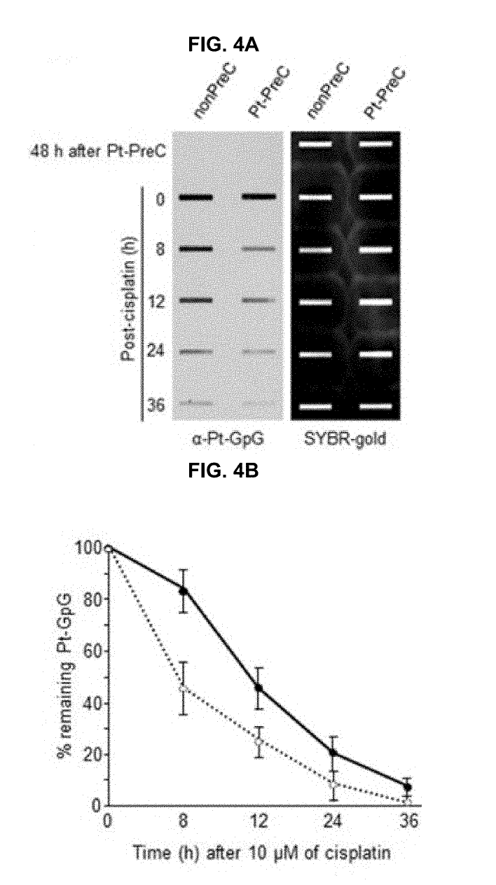

FIGS. 4A and 4B show the enhanced Pt-GpG diadduct removal by Pt-PreC. FIG. 4A shows that cells preconditioned with 5 μM of cisplatin or mock were treated with 10 μM of cisplatin, and Pt-GpG adduct removal rate was measured using immunoslot blotting with Pt-GpG adduct-specific monoclonal antibody. FIG. 4B shows the quantitative analysis for FIG. 4A. The bars and error bars represent the mean±s.d from three independent experiments.

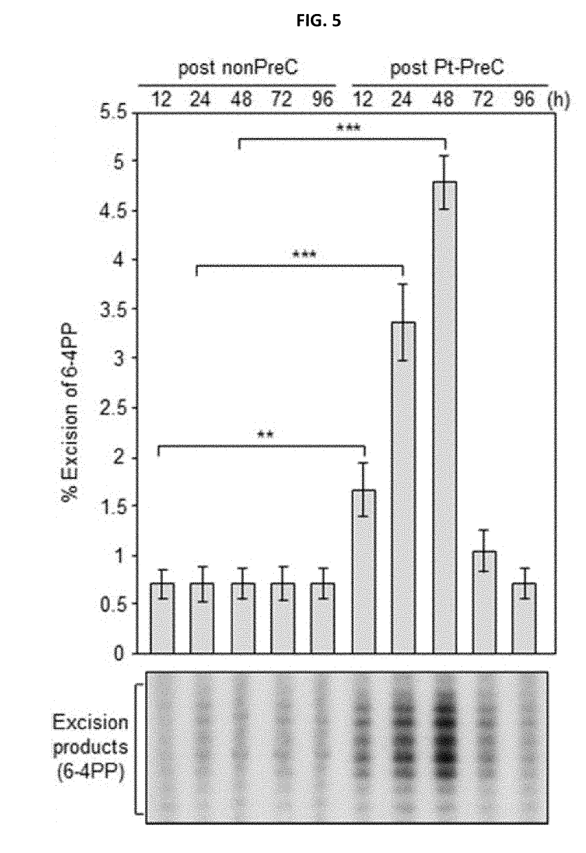

FIG. 5 shows that Pt-PreC enhances NER capacity for 6-4 photoproduct (6-4PP) removal. Dual-incision NER activity assay was performed using isotope-labeled and 6-4PP-containing linear substrate DNA and cell lysates obtained from nonPreC and Pt-PreC cells at the indicated times after PreC. Amount of excision product was used as a measure of the NER capacity of the lysate. Results are presented as mean±SD from three independent experiments. Differences were considered significant at the values of P<0.01 (**) and P<0.001 (***).

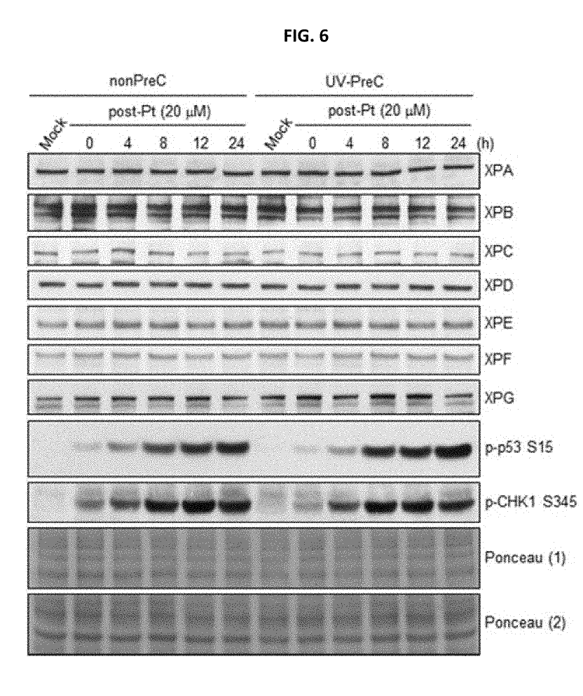

FIG. 6 shows that PreC does not alter the protein expression of core NER factors nor ATR activity. 24 h later of UV-PreC with 5 J/m², cells were treated with 20 μM of cisplatin for 2 h and then cells were allowed to recover for the indicated times. Protein levels of core NER factors (XPA-XPG) and ATR substrate proteins (p-p53 and p-CHK1) were assessed by immunoblotting with the indicated antibodies. Ponceau stained blots from two different gels were used to indicate equal loading of the samples.

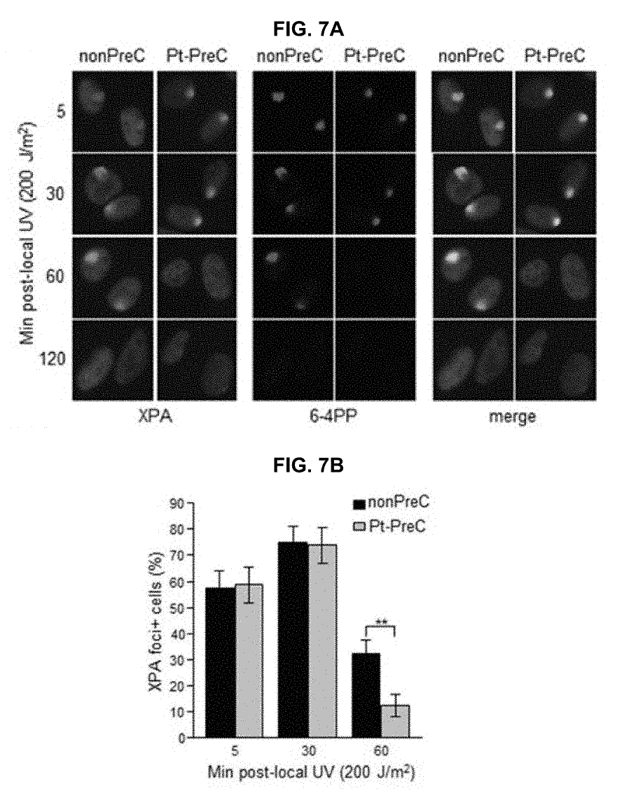

FIGS. 7A and 7B show that PreC accelerates XPA binding to DNA lesions. FIG. 7A shows that 6-4PP removal kinetics and lesion-specific XPA binding from cells preconditioned with nonPreC control or Pt-PreC were monitored after 200 J/m² of local UV irradiation using isopore filter with 5 μm in diameter. FIG. 7B shows that the number of XPA foci-positive cells was calculated among 1000 cells counted. Results are presented as mean±SD from three independent experiments. Differences were considered significant at the value of P<0.01 (**).

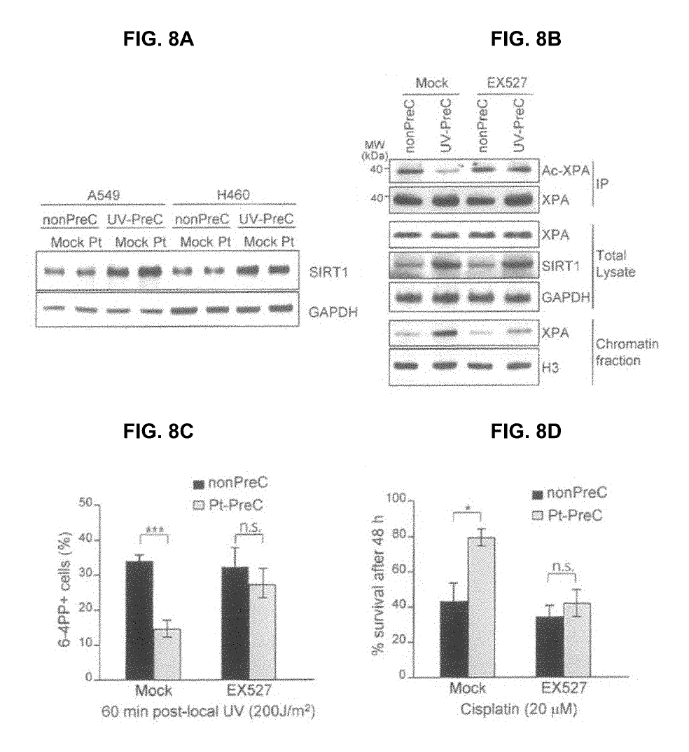

FIGS. 8A to 8D show the upregulation of SIRT1 expression during PreC. FIG. 8A shows that SIRT1 expression from A549 and H460 preconditioned with nonPreC or UV-PreC was assessed by immunoblotting. GAPDH was used as a loading control. FIG. 8B shows that acetylation of XPA was assessed by immunoprecipitation of XPA followed by immunoblotting with anti-acetyl-lysine antibody in the presence or absence of EX527, the SIRT1-specific inhibitor. PreC cells were pretreated with EX527 for 5 h before cisplatin treatment. Histone H3 was used to indicate chromatin-enriched fraction. FIG. 8C shows that 6-4PP repair kinetics from nonPreC or Pt-PreC was measured in the presence or absence of EX527. 60 minutes after local UV irradiation the 6-4PP foci-positive cells were counted from randomly selected 1000 cells in each sample. The bars and error bars represent the mean±s.d from three independent experiments. Differences were considered significant at the value of P<0.001 (***).

### DETAILED DESCRIPTION

The present invention relates to a pharmaceutical composition for lung cancer treatment which, by containing an SIRT1 inhibitor along with a lung cancer therapeutic agent, minimizes the chemoresistance of lung cancer cells to the lung cancer therapeutic agent and exhibits excellent efficacy for lung cancer treatment.

Hereinafter, the present invention will be described in detail.

The pharmaceutical composition for lung cancer treatment according to the present invention contains a lung cancer therapeutic agent and an SIRT1 inhibitor.

In the present specification, a lung cancer therapeutic agent refers to a substance capable of inhibiting the growth, development, activity, etc. of lung cancer cells or inducing death of lung cancer cells.

The lung cancer therapeutic agent according to the present invention is not limited in its type, and any material known in the art as having lung cancer treatment efficacy such as cisplatin, carboplatin, oxaliplatin, or gemcitabine may be used as the lung cancer therapeutic agent. Preferably, the lung cancer therapeutic agent according to the present invention is cisplatin.

In the present specification, an SIRT1 inhibitor refers to a substance capable of inhibiting the expression, activity, etc. of SIRT1 protein or destroying SIRT1 protein. SIRT1 stands for sirtuin (silent mating type information regulation 2 homolog) 1 and is a protein encoded by the SIRT1 gene.

In the present specification, the SIRT1 protein may be a protein derived from a subject to be administered with the pharmaceutical composition of the present invention. For example, the SIRT1 protein is derived from a mammal such as a human, rat, cow, dog, sheep, horse, or pig. In the specific case of when the subject is a human, the SIRT1 protein may have an amino acid sequence of SEQ ID NO: 1.

The inventors of the present invention have devised the present invention based on the finding that an increased level of SIRT1 expression is related to the chemoresistance of lung cancer cells to a lung cancer therapeutic agent specifically by promoting nucleotide excision repair (NER) through the deacetylation of XPA which is involved in NER.

The pharmaceutical composition for lung cancer treatment according to the present invention contains an SIRT1 inhibitor; therefore, the deacetylation of XPA by SIRT1 can be inhibited and the chemoresistance of lung cancer cells to a lung cancer therapeutic agent can be reduced. In this way, lung cancer treatment with maximized efficacy due to minimized chemoresistance can be accomplished.

As the SIRT1 inhibitor of the present invention, a substance known to have inhibitory activity against SIRT1, such as EX527 (6-chloro-2,3,4,9-tetrahydro-1H-carbazole-1-carboxamide), sirtinol (2-[(2-hydroxynaphthalen-1-ylmethylene)amino]-N-(1-phenethyl)benzamide), tenovin-1 (N-[[[4-(acetylamino)phenyl]amino]thioxomethyl-4-(1,1-dimethylethyl)]-benzamide), tenovin-6 (N-[[ [4-[[5-(dimethylamino)-1-oxopentyl]amino]phenyl]amino]thioxomethyl]-4-(1,1-dimethylethyl)-benzamide), cambinol (2,3-dihydro-5-[(2-hydroxy-1-naphthalenyl)methyl]-6-phenyl-2-thioxo-4(1H)-pyrimidinone), salermide (N-[3-[[(2-hydroxy-1-naphthalenyl)methylene]amino]phenyl]-a-methyl-benzeneacetamide), resveratrol (3,4′,5-stilbenetriol), CAY10602 (1-(4-fluorophenyl)-3-(phenylsulfonyl)-1H-pyrrolo [2,3-b]quinoxalin-2-amine), may be used without limitation. One or a combination of two or more of such substances may be used as the SIRT1 inhibitor of the present invention.

In the pharmaceutical composition of the present invention, the content ratio of the lung cancer therapeutic agent and the SIRT1 inhibitor is not particularly limited and may be appropriately adjusted depending on the condition of the subject to be administered, the level of SIRT1 expression, or the like. For example, the SIRT1 inhibitor may be contained in an amount of 0.1 to 200 parts by weight, preferably 1 to 100 parts by weight, and more preferably 10 to 50 parts by weight with respect to 100 parts by weight of the lung cancer therapeutic agent, but the present invention is not limited thereto.

The pharmaceutical composition of the present invention may be formulated into an oral dosage form such as powder, granules, tablets, capsules, a suspension, an emulsion, a syrup, or an aerosol, or into an external preparation, a suppository, or a sterilized injection fluid by a conventional method.

Examples of a carrier, excipient, and diluent that may be contained in the composition of the present invention include lactose, dextrose, sucrose, sorbitol, mannitol, xylitol, erythritol, maltitol, starches, acacia rubber, alginates, gelatin, calcium phosphate, calcium silicates, celluloses, methylcelluloses, microcrystalline celluloses, polyvinylpyrrolidones, water, methyl hydroxybenzoate, propyl hydroxybenzoate, talc, magnesium stearate, and mineral oils. The composition may be formulated into a dosage form using a commonly used diluent or excipient such as a filler, extender, binder, wetting agent, disintegrant, or surfactant.

Solid dosage forms for oral administration include tablets, pills, powders, granules, capsules, and the like and are produced by adding at least one excipient such as starch, calcium carbonate, sucrose, lactose, or gelatin to the composition. In addition to simple excipients, lubricants such as magnesium stearate and talc may be used. Liquid dosage forms for oral administration include suspensions, liquid for internal use, emulsions, and syrups and may contain one or more of various excipients such as wetting agents, sweeteners, fragrances, and preservatives in addition to a commonly used diluent such as water or liquid paraffin.

Preparations for nonoral administration include sterile aqueous solutions, non-aqueous solutions, suspensions, emulsions, freeze-dried preparations, and suppositories. The non-aqueous solutions and suspensions may contain propylene glycol, polyethylene glycol, a vegetable oil such as olive oil, an injectable ester such as ethyl oleate, or the like. As a base material for suppositories, WITEPSOL®, a macrogol, Tween® 61, cacao butter, laurin, glycerogelatin, or the like may be used.

The dosage of the pharmaceutical composition according to the present invention may vary depending on the age, sex, and body weight of the patient. The composition may be administered in an amount of 0.1 to 100 mg/kg once or several times a day and is preferably administered in an amount of 1 to 10 mg/kg once or several times a day. The dosage may also be increased or decreased depending on the route of administration, degree of disease, or the sex, weight, age, etc. of the patient. Therefore, the above-described dosage does not limit the scope of the present invention in any way.

In addition, the present invention provides a method for providing information for determining whether lung cancer cells have developed chemoresistance to a lung cancer therapeutic agent.

The method for providing information for determining whether lung cancer cells have developed chemoresistance to a lung cancer therapeutic agent according to the present invention includes a process of treating lung cancer cells with a lung cancer therapeutic agent and measuring the level of SIRT1 expression.

Since SIRT1 protein is involved in the development of chemoresistance of lung cancer cells to a lung cancer therapeutic agent, information useful in determining whether lung cancer cells have developed chemoresistance to a lung cancer therapeutic agent can be provided by measuring the level of SIRT1 expression.

The lung cancer therapeutic agent may be a lung cancer therapeutic agent known in the art such as cisplatin, carboplatin, oxaliplatin, gemcitabine, or the like. Preferably, the lung cancer therapeutic agent is cisplatin.

The method for measuring the level of SIRT1 expression is not particularly limited to any method and can be performed by any protein expression measurement method known in the art. For example, the level of SIRT1 expression may be measured by the immunoblotting method, but the present invention is not limited thereto.

More specifically, the method for providing information for determining whether lung cancer cells have developed chemoresistance to a lung cancer therapeutic agent according to the present invention may further include the processes of treating the same lung cancer cells with the same lung cancer therapeutic agent for a predetermined time period and then measuring the level of SIRT1 expression once more; and comparing the measured levels of SIRT1 expression. In this case, information on the development of chemoresistance to one type of a lung cancer therapeutic agent that has been used for a certain time period can be provided, and the information can be used in replacing the lung cancer therapeutic agent with another lung cancer therapeutic agent or determining time to administrate an SIRT1 inhibitor.

The predetermined time period may be a time period from when lung cancer cells are treated with a lung cancer therapeutic agent to a time point when it is confirmed that the lung cancer cells express resistance to the lung cancer therapeutic agent, that is, to the estimated time point when the lung cancer cells begin to develop chemoresistance. The time period is not particularly limited and may be from one day to several years.

In another embodiment of the present invention, the method for providing information for determining whether lung cancer cells have developed chemoresistance to a lung cancer therapeutic agent according to the present invention may further include the processes of treating the same lung cancer cells with a plurality of lung cancer therapeutic agents and then measuring the level of SIRT1 expression for each lung cancer therapeutic agent; and comparing the measured levels of SIRT1 expression. In this case, a lung cancer therapeutic agent to which the lung cancer cells are least resistant can be identified, and the information can be used in determining a lung cancer therapeutic agent with low chemoresistance and excellent lung cancer treatment efficacy.

In addition, the present invention provides a method for screening lung cancer therapeutic agents.

The method for screening lung cancer therapeutic agents according to the present invention includes the processes of treating lung cancer cells with a plurality of lung cancer therapeutic agents and then measuring the level of SIRT1 expression for each lung cancer therapeutic agent; and comparing the measured levels of SIRT1 expression.

As described above, SIRT1 is involved in the development of chemoresistance of lung cancer cells to a lung cancer therapeutic agent. Accordingly, the processes of treating lung cancer cells with a plurality of lung cancer therapeutic agents, measuring the level of SIRT1 expression for each lung cancer therapeutic agent, and comparing the measured levels of SIRT1 expression enable the identification of a lung cancer therapeutic agent with the lowest chemoresistance, that is, a lung cancer therapeutic agent associated with the lowest level of SIRT1 expression.

Hereinafter, the present invention will be described in detail with reference to the following exemplary embodiments.

### EXAMPLES

### Materials and Methods

Cell Culture and Cell Viability Assay

A549 and H460 cells (American Type Culture Collection, Manassas, Va., USA) were cultured in Dulbecco's modified Eagle medium supplemented with 10% fetal bovine serum and 1% penicillin-streptomycin. 100% confluent cells were kept for additional four days to completely block cell proliferation and then exposed to the UV light using a germicidal lamp (for immunoslot blotting) or UV crosslinker (for immunofluorescence) emitting primarily UV-C light. A UV-C sensor (UV Products, Upland, Calif., USA) was used to calibrate the fluence rate of the incident light. For immunofluorescence staining, cells were grown on a glass coverslip coated with poly-D-lysine and laminin (BD Biosciences, San Jose, Calif., USA). For assessment of cell viability, CellTiter-Fluor Cell Viability Assay kit (Promega, Madison, Wis., USA) was used as indicated in manufacturer's protocol. Fluorescence plate reader (BIORAD, Hercules, Calif., USA) was used to measure a constitutive protease activity within live cells using a fluorogenic and cell permeable peptide substrate.

Immunoslot Blotting

Genomic DNA was obtained using a QIAamp DNA Mini Kit (Qiagen, Hilden, Germany), and 100 μg (for CPD) or 500 μg (for 6-4PP and platinum [Pt]-GpG adduct) DNA was vacuum-transferred to a nitrocellulose membrane using a BioDot SF Microfiltration apparatus (BIORAD). DNA was crosslinked to the membrane by incubation at 80° C. for 2 h under vacuum. Monoclonal antibodies that recognize CPD (Kamiya, Seattle, Wash., USA), 6-4PP (Cosmo Bio, Tokyo, Japan) and Pt-GpG (Oncolyze, Essen, Germany) were used to detect the amounts of remaining lesions in the genomic DNA. After the immunoslot blot assay, the total DNA amounts loaded onto the membrane were visualized with SYBR-gold staining, and these values were used for normalization.

Dual-Incision NER Activity Assay

Assay of NER activity in the cell lysate toward 6-4PP-containing DNA substrate was carried out. Briefly, 10 fmol of 140-bp duplex with a 6-4PP in the center and ³²P-label at the 5th phosphodiester bond 5′ to the site of the lesion was incubated with 70 μg of lysate in 25 μL of excision buffer at 30° C. for 1 h. The amount of excision product was used as a measure of NER capacity in the lysate. The 6-4PP-containing linear duplex substrate DNA and NER-competent cell lysate were prepared.

Immunoblotting and Immunoprecipitation

Whole-cell lysate prepared in Hea Min Joh et al., Effect of additive oxygen gas on cellular response of lung cancer cells induced by atmospheric pressure helium plasma jet, Scientific Reports 4, Article number: 6638 (2014) was used to determine the levels of proteins. Antibodies used in this study include those against XPA (Kamiya), XPB-XPD (Santa Cruz Biotechnology, Santa Cruz, Calif., USA), XPE, p-p53, p-CHK1, GAPDH, SIRT1, acetyl-lysine (Cell Signaling Technology), XPF, and XPG (both Abcam, Cambridge, UK). For immunoprecipitation of XPA, 1 mg of whole-cell lysate was incubated with 1 μg of anti-XPA conjugated to Protein A/G-agarose beads (Sigma, St. Louis, Mo., USA) for 12 h at 4° C. with rotation. After washing with lysis buffer, proteins were eluted from the beads by boiling in SDS sample buffer and resolved on 10% SDS-polyacrylamide gels. For detection of XPA acetylation, anti-acetyl-lysine was employed.

Local UV Irradiation and Immunofluorescence

Cells preconditioned with cisplatin were irradiated with UV-C at a dose of 200 J/m² through an isopore polycarbonate filter with pores 5 μm in diameter (EMD Millipore). After platinum preconditioning (Pt-PreC), if necessary, cells were treated with 1 μM of specific SIRT1 inhibitor EX-527 (Sigma) for 5 h before local UV irradiation. After incubation for the recovery times the cells were fixed in 4% paraformaldehyde for 15 min, followed by conventional immunofluorescence staining procedures. UV-induced lesions were counter-labeled with anti-6-4PP antibody, and XPA foci-positive cells were counted for quantitative analysis. The images were captured using Nikon imaging software NIS-Elements 4.0.

Statistics

Data were evaluated using Student's t-test, one-way ANOVA with Tukey test, or two-way ANOVA for multiple comparisons as indicated. Results are presented as mean±SD from at least three independent experiments. Differences were considered significant at the values of P<0.05 (*), P<0.01 (**), and P<0.001 (***). Statistical analyses were performed with GraphPad Prism 5.0 software (GraphPad, La Jolla, Calif., USA).

Results

In order to obtain insight into the effect of DNA repair capacity on the mechanism of chemoresistance, we investigated changes in NER activity after treatment of cells with nonlethal doses of DNA-damaging agents. We used two monoclonal antibodies to specifically detect UV-induced CPDs and Pt-GpG adducts, lesions that are the exclusive substrates of NER. To exclude the effect of cell cycle on DNA repair activity, human non-small cell lung carcinoma A549 and large cell lung carcinoma H460 cells were grown to confluence and kept for additional four days to completely block cell proliferation (FIGS. 1A to 1C) before treatment with DNA damaging agents. Several UV doses were applied to measure the repair activity and cell viability. The amount of CPD lesions on genomic DNA was analyzed by immunoslot blotting (FIG. 2A), and cell viability after 24 h of UV exposure was assessed by a fluorescence-based cell viability assay (FIG. 2B). Upon irradiation with 5 J/m² UV, there was no significant decrease of cell number and 24 h was sufficient for complete repair of CPDs. However, irradiation of cells with more than 5 J/m², including 10 or 20 J/m², resulted in substantial decrease in cell number, and CPDs still remained on genomic DNA at 24 h after UV irradiation. Based on these results, we chose 5 J/m² of UV dose as a repairable and nonlethal condition for activation of DNA damage response in lung cancer cells, which behaved like recurrent cancer or chemoresistant cells after primary chemotherapy with DNA-damaging agents. We termed this condition as “preconditioning” (PreC) and examined whether it affected subsequent DNA repair activity evoked by cisplatin treatment at a lethal concentration of 10 μM. As shown in FIGS. 2C and 2D, UV-PreC facilitated repair of subsequent Pt-GpG adduct compared to the nonpreconditioned control (nonPreC). In the inverse experiment, we preconditioned cells with a nonlethal concentration of cisplatin (5 μM) and then investigated repair of the UV-induced CPDs. As expected, repair of CPDs caused by 10 J/m² UV required more time than those induced by 5 J/m² UV (FIG. 3A, lanes 1 and 2). This pattern was also observed when cells were preconditioned with cisplatin (FIG. 3A, lanes 4 and 5). However, the kinetics of CPD repair after the same dose of UV were much faster when cells had been preconditioned with cisplatin (FIGS. 3B and 3C), which is similar to the effect of UV-PreC shown in FIGS. 1A to 1C.

Next, we measured Pt-GpG removal rate following Pt-PreC. As shown in FIG. 4A, there was no remaining Pt-GpG adduct after 48 h of Pt-PreC with 5 μM of cisplatin. Meanwhile the repair kinetics upon following cisplatin treatment was much faster in Pt-PreC than nonPreC cells (FIG. 4B). These results suggest that NER activity may be upregulated by PreC with a nonlethal dose of DNA-damaging agent. To test this hypothesis we measured the cell's NER capacity at specific times from 12 h to 96 h after PreC. To this end, we used an in vitro dual-incision assay, for which we prepared DNA substrate containing UV-induced 6-4PP, which is a better substrate than CPD, and cell lysate prepared at various time points after PreC. FIG. 5 shows that the lysate of nonPreC cells had no time-dependent effect on dual-incision activity, whereas the lysate of Pt-PreC cells showed changes in NER capacity depending on the duration of time after PreC. NER activity started to increase 12 h after PreC and peaked at 48 h, at which time the lesions were completely repaired. However, the enhancement of NER capacity by PreC was no longer detected 72 h after PreC (FIG. 5).

To decipher the mechanism underlying enhancement of NER capacity by PreC, we first analyzed the levels of core NER factors XPA through XPG at 24 h after PreC and compared this with levels in nonpreconditioned controls because some previous reports demonstrated increase of core NER factors including XPA and XPF during adaptive response. As shown in FIG. 6, however, there was no significant change in the expression of NER factors regardless of PreC. Next, we analyzed ATR kinase activity indirectly by monitoring the level of phosphorylation of its substrate proteins p53 and CHK1. ATR is known to augment NER activity by phosphorylating and, thus stabilizing, XPA in response to DNA damage. The result indicates that UV-PreC had no effect on ATR activity as no significant alteration in phosphorylation of p53 or CHK1 was detected after PreC (FIG. 6). In addition, similar phosphorylation profiles were obtained from nonPreC and UV-PreC cells, which implies that ATR activity had not been altered by PreC.

XPA is the key rate-limiting factor for NER. However, given that PreC had no effect on XPA protein level or ATR activity, we next examined the effect of PreC on XPA mobility to damaged DNA using local UV irradiation. XPA foci at locally-exposed sites strongly coincided with 6-4PP lesions (FIG. 7A). As similar as shown in immunoslot blot data in previous figures, Pt-PreC accelerated the 6-4PP removal than nonPreC control, as demonstrated by more rapid disappearance of 6-4PP signal (FIG. 7A). For a quantitative analysis we counted the number of XPA foci-positive cells and found no difference between nonPreC and Pt-PreC within 30 min of recovery time (FIG. 7B). However, at 60 min after UV exposure approximately 3 times less XPA foci-positive cells were detected in Pt-PreC, indicating that the Pt-PreC may modulate the efficient XPA recruitment on DNA lesions followed by a robust repair and possibly conferring resistance to toxic DNA damage. Because SIRT1, a histone deacetylase, has been implicated recently in the NER pathway by virtue of deacetylating XPA and thus enhancing NER activity, we measured the level of SIRT1. Interestingly, UV-PreC cells showed increased levels of SIRT1 compared to nonPreC control (FIG. 8A). To verify the acetylation status of XPA we immunoprecipitated XPA and determined the acetylation level with anti-acetyl-lysine antibody. The result indeed indicated a decrease in acetylation level of XPA with UV-PreC, which is immediately reversed by treatment of SIRT1 inhibitor EX527 (FIG. 8B). To confirm the role of SIRT1 in the PreC effect, we pretreated cells with the specific SIRT1 inhibitor EX-527 before treatment of cisplatin following the UV-PreC and investigated XPA loading to chromatin. UV-PreC-induced enhancement of XPA chromatin loading was reduced in the presence of EX527, implying that the SIRT1 regulated XPA acetyl status may contribute XPA sensitivity to DNA lesions. The PreC-induced repair capacity was also compromised by SIRT1 inhibition (FIG. 8C), which implies that upregulation of SIRT1 is the major mechanism in PreC-induced NER potentiation.

These results indicate that SIRT1 expression was increased more in PreC compared to in nonPreC, and that the inhibition of SIRT1 activity induced death of p53-associated cells. Therefore, it can be seen that reduction in SIRT1 activity may be helpful in cancer treatment.

1. A pharmaceutical composition for lung cancer treatment comprising a lung cancer therapeutic agent and an SIRT1 inhibitor.

2. The pharmaceutical composition of claim 1, wherein the lung cancer therapeutic agent is one or more selected from the group consisting of cisplatin, carboplatin, oxaliplatin, and gemcitabine.

3. The pharmaceutical composition of claim 1, wherein the SIRT1 inhibitor is one or more selected from the group consisting of EX527, sirtinol, tenovin-1, tenovin-6, cambinol, salermide, resveratrol, and CAY10602.

4. A method of providing information for determining whether lung cancer cells have developed chemoresistance to a lung cancer therapeutic agent, the method comprising:
treating lung cancer cells with a lung cancer therapeutic agent; and
measuring a level of SIRT1 expression.

5. The method of claim 4, wherein the lung cancer therapeutic agent is one or more selected from the group consisting of cisplatin, carboplatin, oxaliplatin, and gemcitabine.

6. The method of claim 4, further comprising:
treating the same lung cancer cells with the same cancer therapeutic agent for a predetermined time period and then measuring a level of SIRT1 expression once more; and
comparing the measured levels of SIRT1 expression.

7. The method of claim 4, further comprising:
treating the same lung cancer cells with a plurality of lung cancer therapeutic agents and then measuring a level of SIRT1 expression for each of the plurality of lung cancer therapeutic agents; and
comparing the measured levels of SIRT1 expression.

8. A method for screening lung cancer therapeutic agents, the method comprising:
treating lung cancer cells with a plurality of lung cancer therapeutic agents and then measuring a level of SIRT1 expression for each of the plurality of lung cancer therapeutic agents; and
comparing the measured levels of SIRT1 expression.

9. The method of claim 8, which identifies a lung cancer therapeutic agent with a lowest level of SIRT1 expression as a lung cancer therapeutic agent with a lowest chemoresistance.

10. A method for treating lung cancer in a subject in need thereof, the method comprising administering a therapeutically effective amount of the pharmaceutical composition of claim 1 to the subject.

11. The method of claim 10, wherein the lung cancer therapeutic agent is one or more selected from the group consisting of cisplatin, carboplatin, oxaliplatin, and gemcitabine.

12. The method of claim 10, wherein the SIRT1 inhibitor is one or more selected from the group consisting of EX527, sirtinol, tenovin-1, tenovin-6, cambinol, salermide, resveratrol, and CAY10602.

13. The method of claim 10, wherein the lung cancer therapeutic agent is cisplatin, and the SIRT1 inhibitor is EX527.
